(19) United States
(12) Patent Application Publication
(10) Pub. No.: US 2010/0324415 A1
(43) Pub. Date: Dec. 23, 2010

(54) PLATFORM FOR DETECTION OF TISSUE STRUCTURE CHANGE

(75) Inventors: Darrel D. Drinan, San Diego, CA (US); Carl F. Edman, San Diego, CA (US); Naresh C. Bhavaraju, San Diego, CA (US)

Correspondence Address: KNOBBE MARTENS OLSON & BEAR LLP, 2040 MAIN STREET, FOURTEENTH FLOOR, IRVINE, CA, 92614, US

(73) Assignee: PhiloMetron, Inc., San Diego, CA (US)

(21) Appl. No.: 12/754,490

(22) Filed: Apr. 5, 2010

Related U.S. Application Data
(63) Continuation of application No. 11/837,357, filed on Aug. 10, 2007, now abandoned.
(60) Provisional application No. 60/837,423, filed on Aug. 12, 2006.
(60) Provisional application No. 60/848,079, filed on Sep. 29, 2006.

(51) Int. Cl. A61B 6/00

(52) U.S. Cl. 600/425

(57) ABSTRACT

Aspects include methods and apparatuses for determining change over time in one or more measured regions of a body using a plurality of data sets obtained by analysis of applied signals to said region. The method may include transmitting and receiving one or more of electromagnetic wave signals, applied acoustic wave signals and electrical signals transmitted through or reflected off of a portion of the measured body region.

### CROSS-REFERENCE TO RELATED APPLICATIONS

This application is a continuation of U.S. application Ser. No. 11/837,357 filed Aug. 10, 2007 which claims priority under 35 U.S.C. 119(e) to U.S. Provisional Application No. 60/837,423, filed on Aug. 12, 2006, entitled PLATFORM FOR DETECTION OF HYDRATION AND/OR STRUCTURAL CHANGE IN MAMMALIAN ORGANISMS, and U.S. Provisional Application No. 60/848,079, filed on Sep. 29, 2006, entitled PLATFORM FOR DETECTION OF CONTENT AND/OR STRUCTURAL CHANGE OF TISSUE IN MAMMALIAN ORGANISMS.

### BACKGROUND OF THE INVENTION

1. Field of the Invention

The invention relates to medical diagnostic equipment. In particular, the invention relates to monitoring systems to assess content and/or structure of tissue.

2. Description of the Related Art

Methods for measuring subsurface structures and content exist that utilize electromagnetic, infrared, acoustic and/or electrical waves/signals. The electromagnetic form can be RF or microwave frequency radiation. This electromagnetic measurement may utilize time shifts of all or a portion of the transmitted wave to provide information regarding the tissue content or structure. The reflected intensity of these waves likewise provides information regarding internal structures.

Accordingly, a variety of systems and methods have been proposed to image or detect internal structures with RF or microwave electromagnetic radiation. For example, U.S. Pat. No. 5,829,437 to Bridges teaches the use of backscattered radio waves reflected by change in dielectric constant resulting from the differential composition of tissues to detect abnormalities, e.g. tumors, in tissue. Another example includes U.S. Pat. No. 7,089,047 which measures fat depth with microwaves.

Electromagnetic radiation has also been used to measure non-structural physiological features. In its most common embodiment, such measurements are employed to detect and characterize movement. For example, McEwan (U.S. Pat. No. 5,573,012) teaches the use of pulse-echo radar in repetitive mode to measure heart motion. Likewise, Sharpe et al. teach the use of radio frequency waves for non-contact measurement of movement of the surface of the body from which respiration and heart rate may be determined (U.S. Pat. No. 4,958,638). Another example is provided in U.S. Pat. No. 6,849,046 to Eyal-Bickels et al. where a system is described that measures hydration levels of a subject (water content) with transmitted RF energy at 2.45 and 40.68 GHz.

### SUMMARY OF CERTAIN INVENTIVE ASPECTS

Although RF and microwave energy has been used to detect internal physiological structures, detection of changes in structure based on detection of changes in backscattered or transmitted RF or microwave energy in the context of relatively long term periodic or continuous monitoring has not been performed, nor have the benefits of this approach been appreciated. There exists a need for an accurate, objective and convenient monitoring system to assess change in the structure of tissue within mammalian organisms. Assessment of change in status provided by the inventions described herein may permit more effective therapeutic interventions and therapies to be applied in response to the measured change in status.

In one embodiment, the invention provides a method of detecting a physiological condition in a portion of a subject. This method comprises applying electromagnetic waves having frequencies within a range of about 1 GHz to about 100 GHz to a region of the subject at a plurality of points in time, and deriving a plurality of data sets from measured parameters of interaction between the region of the subject and the radio frequency or microwave frequency electromagnetic waves at the plurality of points in time, wherein the content of the data sets is dependent at least in part on tissue structure in the region at the plurality of points in time. The plurality of data sets are compared to detect a change over time in the interaction between the region of the subject and the electromagnetic waves. A change in tissue structure is detected in the region of the subject based at least in part on the comparing.

In another embodiment, a method of determining detecting a physiological condition in a portion of a subject comprises applying electromagnetic waves residing within a frequency range from about 1 GHz to about 100 GHz to a region of the subject at a plurality of points in time as well as applying a different form of energy to the region of the subject at a plurality of points in time. The method also includes deriving a first plurality of data sets from measured parameters of interaction between the region of the subject and the electromagnetic waves at the plurality of points in time and deriving a second plurality of data sets from measured parameters of interaction between the region of the subject and the different form of energy at the plurality of points in time. The first and second plurality of data sets are compared to detect a change over time in the interaction between the region of the subject and the electromagnetic waves and to detect a change over time in the interaction between the region of the subject and the different fours of energy. A change in tissue structure is detected in the region of the subject based at least in part on the comparing.

In another embodiment, a system for measuring change over time in tissue structure in a body region comprises at least one antenna and circuitry coupled thereto configured to transmit and receive electromagnetic wave signals, circuitry configured to convert the signals into one or more data sets, and a comparator subsystem for performing mathematical calculations on at least two data sets for the determination of change in the tissue structure of the measured body region.

In another embodiment, a system for measuring change over time in tissue structure in a body region comprises an applicator of about 1 GHz to about 100 GHz electromagnetic energy, a detector configured to detect parameters of interaction between the tissue and the electromagnetic energy, an applicator of another form of energy; and a detector configured to detect parameters of interaction between the tissue and the other form of energy. In this embodiment, the applicators are housed or supported by a common structure.

### BRIEF DESCRIPTION OF THE DRAWINGS

FIG. 1 is a flow chart of a method of detecting structural change according to one embodiment of the invention.

FIG. 2 is a schematic representation of an embodiment of a measuring system in accordance with one embodiment of the invention.

FIG. 3 is a block diagram of a measuring system in accordance with one embodiment of the invention.

FIG. 4 is an illustration of a body limb section with a UWB device such as, for example, the device illustrated in FIG. 1, affixed to a surface of tissue.

FIG. 5 is an illustration of a slowing effect of a 120 ps UWB pulse due to fluid change in tissue.

### DETAILED DESCRIPTION OF CERTAIN INVENTIVE EMBODIMENTS

The invention will now be described in association with the above described Figures, where like numerals refer to like elements. Furthermore, in the discussion that follows, the following definitions are applicable.

Body—a portion or all of the torso, arms, legs, head or neck of a subject, including any or all of the exterior and/or interior of the body.

Bioparameter—a physical factor associated with a body or user able to be measured and quantified.

Subject—a mammal being measured using the method or devices of the invention.

General Aspects of the Inventions

Aspects of the invention relate to structural change detection based on changes in a measured interaction between a portion of a measured subject and applied electromagnetic waves. Accordingly, one aspect of the invention provides a method of periodically or continuously interrogating a tissue region of interest with electromagnetic waves to determine if the structure of the tissue has changed over time, or is different than a baseline structure that produced a baseline interrogation “signature” stored in memory. Various forms of electromagnetic waves can be compared to the baseline with the use of a comparator for determination of regional changes over time of body structures of mammalian bodies. The changes may be on the surface, beneath the surface or span the surface to the subsurface of the measured body region. In one embodiment of the invention, electromagnetic waves based upon ultra wideband (UWB) radar technology are used. In alternate embodiments of the invention, bands of one or more other frequencies may be applied to the body for the determination of change. The measured values arising from the signal are incorporated into a data set representing the status of the region at a certain time point. These data sets may then be stored for future comparison to data sets of measurements taken at other times to determine change in the properties or characteristics of a tissue or region being measured. It is an important aim of the invention to detect structural changes in the tissue (e.g. tumor growth, vascular system changes) rather than merely non-structural chemical content changes or non-structural physical changes such as temperature change. It will be appreciated, however, that content changes and the like can in some cases cause structural change. As described below, therefore, it can be beneficial in some embodiments to use more than one interrogation modality to provide information about whether measured changes in electromagnetic wave interaction is due to structural change, non-structural change, or both.

In another aspect of the invention, circuitry, a power supply and a transceiver for delivering and receiving the electromagnetic energy for construction of the measurement data set are contained either wholly or in part in a structure, e.g. a patch, affixed to or implanted within the body. In an alternate embodiment of the invention, the circuitry and transceiver for measuring signals are contained in a structure not affixed to the body. The non-affixed structure may be hand held or otherwise supported to permit the measurement activity. Such supports may include inclusion of one or more devices into fixed structures present in the living space of measured subject, i.e. within beds, closets, bathrooms, etc., thereby permitting unobtrusive measurements to be obtained periodically without disruption of lifestyle or activities.

In some embodiments of the invention, guidance for measurements and/or comparator activities may be utilized to aid determination of location of specific regions or targets of measurements. Guidance may be provided in the form of comparative mapping utilizing anatomical landmarks and/or active or passive fiducial marks or devices.

The measurement devices may store information relating to the measurement event, e.g. time, or signal data, for later retrieval and analysis. In addition, the measurement devices may have in part or in whole comparators allowing processing of raw data, e.g. mathematical transforms, for the purpose of facilitating storage, transmittal or display of the signal data.

In one embodiment of the invention, the electromagnetic data sets are combined and utilized by the comparator with other physiological measurements, e.g. optical, electrical or mechanical, to provide greater insight into changes of the body region being measured. These other physiological measurements may include, but are not limited to, temperature, body weight, bioelectric impedance, or optical, e.g. infrared, measurements. These additional measurements may be especially valuable in detecting changes in content of tissue (e.g. water content or hydration) that will cause changes in the RF or microwave signals but that are not related to structural changes in the tissue.

In other embodiments of the invention, the data set may be combined and used by the comparator with other measurements of physiological status, including but not limited to, nutritional and/or medical history, subjective responses to questions, diagnostic test results, e.g. blood composition analysis or urinalysis, to provide fuller assessment of possible changes in the region being measured.

In still other embodiments of the invention, one or more of the applied signals, e.g. radio frequency, optical or impedance, may be utilized for purposes of identification either of applied materials or devices on or within the mammalian body and/or for the purpose of identification of the mammalian body. This identification may be useful for numerous reasons, including but not limited to, ensuring correct management of applied therapies, or the tracking of devices and/or persons. Either existing structures within the body or applied materials may supply features necessary to ensure the correct level of identification or alternatively, additional markers or structures may be added for this purpose.

In some embodiments of the invention, the collected data sets may be transmitted by wireless or wired means to data collection units. Upon reception of one or more data sets, a data collection unit may display the data set(s) and/or perform comparator activities upon the received data set and display the results of this activity. Such data collection units may collect data measurement sets from one or more measurement devices. In addition, such data collection units may be used to display data set values, or mathematical transforms of said data sets, including trending and combinations with other sensor or input data. In yet other embodiments of the invention, said data sets or mathematical transforms of said data sets may be relayed to yet other data collection units or remote data management systems for data storage, display or additional analysis, e.g. population based or group trend analysis.

The invention described herein includes methods and devices for determining the effective change in electrical (including but not limited to conductivity, permittivity, permeability), physical (including but not limited density, viscosity etc) or chemical (including but not limited Cl−, Na+, K+ ions) properties of one or more inspected regions of tissue and/or bodily fluids. Change in these properties may correspond to changes in tissue structure such as changes in relative proportional volume of various heterogeneous components that comprise a living mammalian body. This change in structure results in a change in the time of flight of an applied electromagnetic signal through the inspected body region and/or a change in the power attenuation of the signal of the applied signal through this inspected body region. These changes in signal are useful to monitor relative changes in structures of dermal, transdermal or subdermal, or deep tissue regions of mammalian bodies. These changes may be, but are not limited to, changes associated with: normal bodily functions or processes, e.g. growth or development of tissues or organs, detection or development of disease states (e.g. detection of hypertension), the growth of tumors, or the monitoring of therapeutic regimens upon the measured region or aspects of the region (e.g. allograft or transplanted organ rejection). In certain embodiments, the relative change may be calibrated to allow quantification of this change, either in size and/or composition of the inspected region or parts thereof.

In an embodiment of the invention, the measurement device (transmitter, sensors plus circuitry) is affixed to a body site by means of adhesive. In alternate embodiments, the device or sensors may be affixed to the body by means of straps, clothing items or by anchoring directly to or beneath the skin or held against the skin by hand or other supports. In still other embodiments, the measurement device is not in direct contact with the surface of the skin. The scope of the invention is not limited to any one body site or means of affixing the device to the body or adjacent to or not in direct contact with the body.

In some embodiments of the invention, a second device, e.g. a data collection and display unit, may be in communication with the measurement device. Such communication may be bi-directional and include optical, electromagnetic, mechanical, electrical, and/or acoustic means. In one such embodiment of the invention, the antenna and/or aspects of the transmission circuitry utilized for measurements within the measurement device may also be employed for communication of the data set, or mathematical transforms of the data set, to one or more data collection units.

Comparator activities may be performed by systems located in the measurement device, located in the data collection unit, located in a remote data management system or collocated in any of these devices.

The monitoring period may extend from a relatively short period, e.g. fractions of seconds or minutes, to a more extended period, e.g. hours or days, dependent upon the purpose of monitoring and/or user acceptance. In alternative embodiments, the monitoring period may be periodic, e.g. for an hour or two distributed over a day or for a day or two distributed over months. The needs for such monitoring periods again may be set by the purpose of monitoring and/or user acceptance.

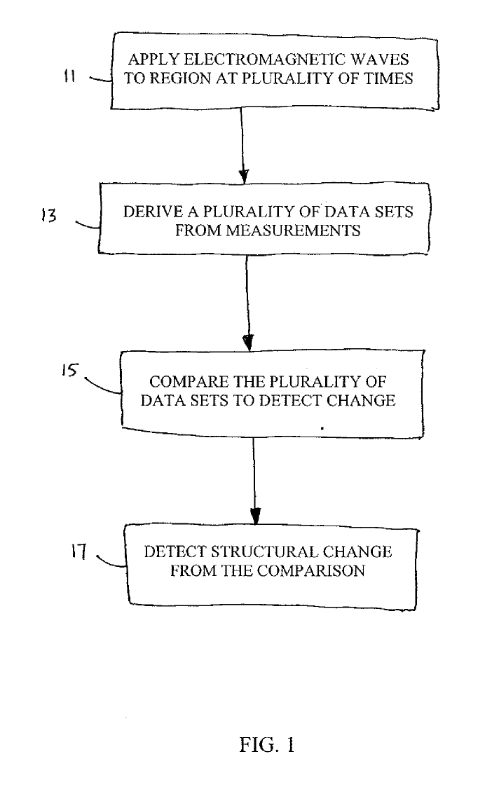

In accordance with the above, a method of detecting structure change is illustrated in FIG. 1. In this method, at block 11, electromagnetic waves are applied to a region of interest on a body at a plurality of different times. At block 13, a plurality of data sets are obtained form a plurality of measurements made at different times of the interaction of the electromagnetic waves with the tissue. At block 15, the data sets are compared to detect changes in the interaction. At block 17, structural changes are detected from the comparison of the data sets.

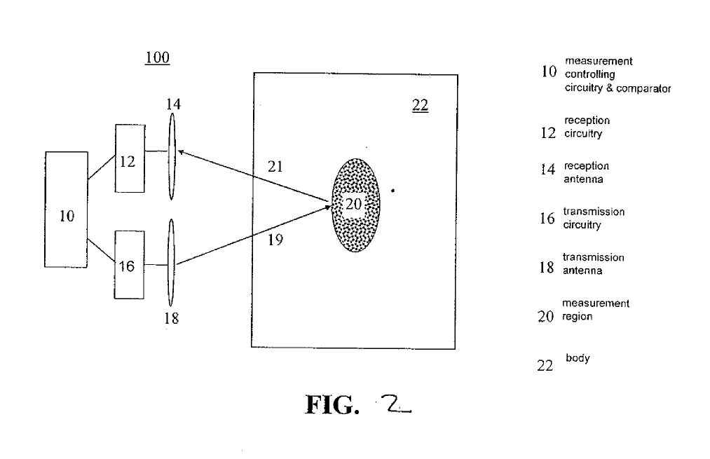

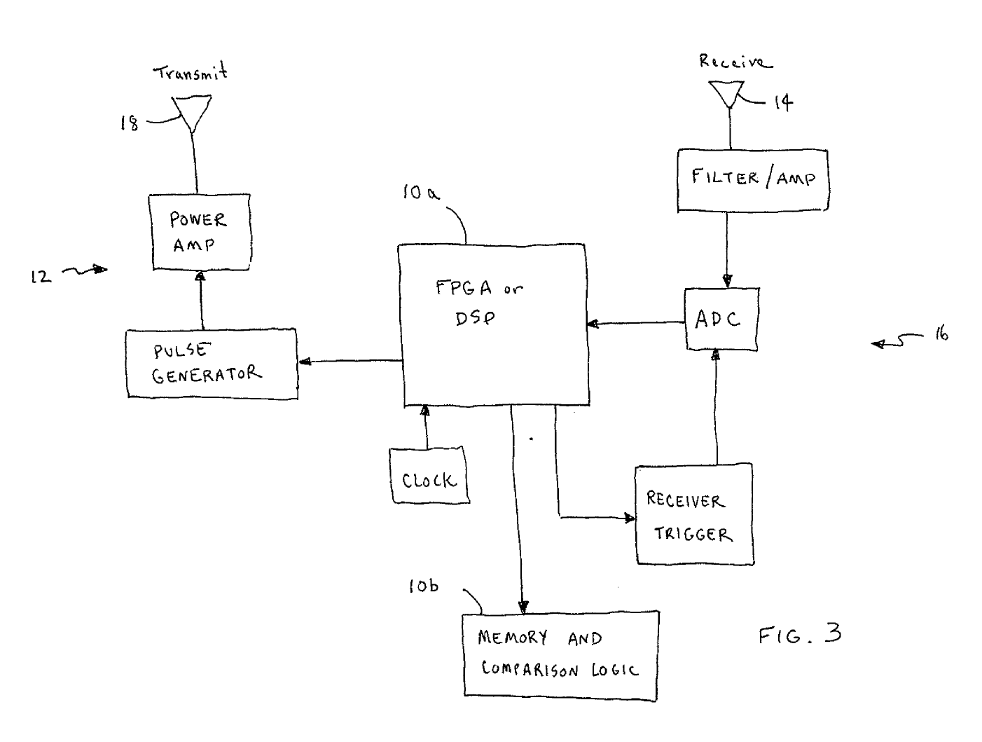

One form of electromagnetic wave that can be employed in embodiments of the invention is ultra wideband (UWB) radiation. However, the scope of the invention is not restricted to UWB but may also employ other forms electromagnetic waves, e.g. discrete bands of other frequencies. The circuitry, power sources and transmission requirements of UWB are well known to those skilled in the art of radio electronics. One representation of components for the measurement device is shown in FIGS. 2 and 3. As shown, a control element 10 is responsible for controlling the initiation of the electromagnetic wave signal, and the subsequent conversion of the reflection of the electromagnetic signal into a measurement data set. As shown in FIG. 3, the control element 10 may comprise a programmable gate array or digital signal processor 10a. The control element 10 controls transmission circuitry 16 which transmits an input signal to a transmission antenna 18. The control element 10 may control various parameters of the transmitted UWB signal including the frequency, the duration, the power level as well as other parameters. The transmission circuitry 16 is electrically connected to the transmission antenna 18 which amplifies and directs the UWB signal to a measurement region 20 within a body 22. The measurement region 20 may be any general point of interest in any body. The transmitted signal 19 is shown reflecting off the measurement region 20 where the reflected signal 21 is then received by the reception antenna 14 and the reception circuitry 12. The control element also controls the reception circuitry 12.

In the example shown in FIG. 2, the transmitted signal 19 is shown to be reflected off of the measurement region 20. However, in some cases, the transmitted signal 19 may pass through the measurement region 20 and reflect off of another region, e.g., a bone. In addition, multiple reflected signals 21 may be received by the reception antenna 14 where the multiple reflections may be reflected off of multiple layers of tissue. The presence of a newly reflected signal at one time point that was not present in the data collected at a previous time point may be an indication of a new structure within the search region 20.

In addition to controlling the transmission circuitry 16 and the reception circuitry 12, the controlling element 10 also contains the comparator for determination of change in the measured region. Also shown are the corresponding signal transmission and reception circuit elements 16 and 12, respectively, as well as corresponding transmission and reception antennas 18 and 14, respectively, for the transmission and reception of the signals. A variety of arrangements of components may be employed in execution of the method of the invention, e.g. combined functionality within one circuit module or the use of a single transmission/reception antenna, and the method of this invention is not limited to any one method of execution. Likewise, in certain embodiments of the invention, a plurality of devices, or components, e.g. transmitters and/or receivers, may be employed to provide additional information of the region being measured.

In contrast to prior art electromagnetic interrogation/imaging systems, preferred embodiments of the invention compare data sets taken at different times to detect structural change, rather than attempting to detect or image a tissue region for immediate characterization at the time the measurement is made. To this end, a comparator subsystem 10b of the control element 10 may include both memory and logic circuitry to store data, thresholds, baselines, etc. and determine change between data sets. The comparator subsystem can be incorporated integrally with a single control logic circuit or can be separately provided and/or remote from the control circuitry for obtaining the measurement data.

Determinations of change may include the use of input threshold values, threshold set points determined by change from baseline value (or representation of one or more data points indicative of a baseline value). Alternatively, such comparator functions may include the use of mathematical transformations such as noise signal subtraction, or rolling or moving averages to determine trends in the data set and to allow adjustment for data taken at different points within the day, e.g. diurnal variation adjustment. Because a variety of non-structural phenomena such as movement, eating patterns, position, and the like can affect measurements without indicating actual structure changes, the data sets can be adjusted or compensated for known contributions of these kinds. In some embodiments, additional sensors such as timers, accelerometers, and other sensing devices can be included to facilitate detection of these factors and removing them from the structural change signal that is being measured. Still other forms of comparator activity may review populations or groups of data for the determination of initial baseline values and for trends of data sets or groups. The results of such comparator activity may be displayed graphically, e.g. showing baseline values and relative change from these values over time, including the projection of future trends.

In addition, the comparator subsystem may incorporate other factors, such as input parameters associated, e.g. weight, height, age, gender, disease status and medication history, fitness level, body site of device application, ethnicity, etc. or parameters derived from algorithms arising studies allowing further definition of change and/or the magnitude of such change. Such parameters may include factors relating subjective user or clinician perception of change to the measured bioparameter, either upon the event or periodically, e.g. daily.

In yet other embodiments of the invention, the comparator subsystem may include other factors including data derived from other measured bioparameters such as levels of circulating hormones or metabolites or activity measurements, or data obtained from environmental sensors, e.g. relative humidity, ambient temperature, etc. This invention may employ combinations of these as well as other factors and the scope of the invention is not limited to those factors and mathematical routines described herein.

The comparator subsystem may reside in a variety of locations. In one embodiment of the invention, the comparator may be contained either in part or in whole within circuitry necessary to acquire the data set. In other embodiments of the invention, the comparator may be located in a separate unit connected by wires to the sensors and/or sensor circuitry, e.g. the transceiver. In such embodiments, the sensors or sensor circuitry may have identities separate and distinct from the unit comprising in part or in whole the comparator activities. In still other embodiments of the invention, the location of comparator activities may shift in order to facilitate data analysis, e.g. to accommodate greater sophistication and/or larger data sets, or for other purposes, e.g. power management of devices, data collection units, etc.

In yet other embodiments of the invention, measured values or mathematical transforms, e.g. averages, or percentage change, of one or more measured bioparameters are transmitted through wireless means to a separate unit not necessarily located on the body. This separate unit may contain either in portion or entirely the appropriate elements and circuitry, e.g. transmission means, data storage and mathematical calculator functions and routines, to perform the comparator activities. Additional forms and locations for the comparator are readily conceivable and the scope of the invention is not limited to those described herein.

Upon determination of a change in status in the measured region, as well as the possible determination of the magnitude of such events, the comparator may be instructed to display such events to the subject, caregiver or third party individual. Display of any changes may also include the notification of no change as compared to all or a portion of the data set. Such displays may include visual displays, e.g. anatomical maps of the region including two dimensional and three dimensional representations, blinking or multicolored lights, numeric indices, graphs, or charts, audible sounds or mechanical forms, e.g. vibrations. Such displays may be located on the on-body measurement device, a local data collection unit or at a remote location connected to either the on-body measurement device or a local data collection unit by wireless or wired means.

In addition to possibly displaying the change, the comparator may store the event description, including date/time, magnitude and user identification, in a data storage. Such data storage may include electronic memory, magnetic tape or disk memory, optical memory or other form of retrievable memory. Such data may be retrieved from storage either on command or periodically from the memory storage. In certain embodiments, such retrieval may be through wireless means, e.g. infrared or radio frequency based data transmission or in other embodiments, such retrieval may be through wired means, e.g. by use of a docking station attached to a computer or by serial cable linkage to a computer.

UWB systems transmit signals across a much wider frequency than conventional systems. The amount of spectrum occupied by a UWB signal, i.e. the bandwidth of the UWB signal can be about 25% of the center frequency or more. Thus, a UWB signal centered at 2 GHz could have a bandwidth of about 500 MHz and the bandwidth of a UWB signal centered at 4 GHz could be about 1 GHz. The most common technique for generating a UWB signal is to transmit pulses with durations less than 1 nanosecond. In preferred embodiments, at least some of the applied electromagnetic waves are within a frequency range between about 1 GHz to about 100 GHz.

Using a UWB system, one can non-invasively and without touching the surface of the skin, evaluate the gross anatomy of internal organs in the body. UWB uses short pulses and reflections, i.e. reflected signal strength and/or delay in signal time of flight, of these pulses from different layers inside the body to obtain morphological information of the anatomical structures and their movement deep inside the body. Some typical applications that could utilize UWB signals for evaluation are heart wall movement, respiration, and obstetrics.

The main advantages of UWB over other technologies such as ultrasound are the following:
UWB signals do not need to be in contact with the skin because they are less affected by transmission through air than sound waves.
UWB signals do not attenuate in the bone and hence can obtain information inside cavities covered by bone, such as the brain.
UWB signals can be used to collect data through non-conductive material such as cloth, bedding, hazmat suits, or body armor.
UWB signals can be realized with a small number of inexpensive components enabling low power, portable applications.

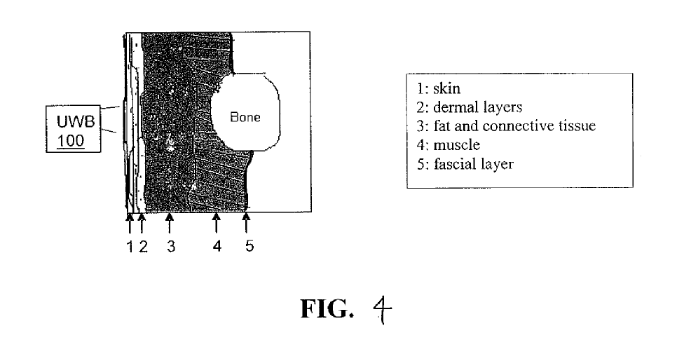

As noted above, the UWB measurement system detects the changes in the structure of the tissue by examining signal reflections from the tissue layers. For example, a transmitter sends a UWB pulse and then receives the pulse reflections from the different layers of the tissue. Because the speed of the UWB radar is different in different types of tissues (e.g. the signal propagation is approximately 2.25 times faster in fat than in muscle) and the layers of tissue are at different depths, the reflections from the tissue layers reach the receiver at different times and have different amplitudes. Further, the attenuation of the signal as it travels through the tissue gives the density information of the tissue. For example, when placed next to a limb including a bone, as shown FIG. 4, the UWB radar measurement system 100 can assess any changes in volume in the various layers by measuring the time delay of the received pulses and their amplitude relative to the corresponding parameters during baseline evaluation. The layers shown in FIG. 4 include a skin layer 1, a dermal layer 2, a fat and connective tissue layer 3, a muscle layer 4 and a fascial layer 5. Other layers may also be evaluated.

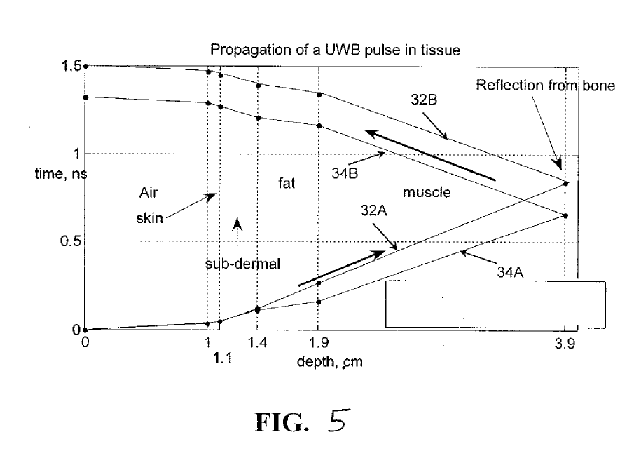

FIG. 5 illustrates an example of how a UWB pulse travels through tissue and how structural change in the tissue can affect the amplitude and delay of the reflected pulses. This is because different types of tissue with different structures have different permittivity, resulting in slowing of the pulse by different amounts depending on relative tissue volumes and positions. Additionally, there are typically amplitude changes in the reflected waves.

As shown in FIG. 5, a first baseline measurement is made and is depicted as a transmitted signal 34A and a reflected signal 34B. The transmitted signal 34A starts at a position 1 cm above the skin (listed as a depth of 0.0 on the horizontal axis). The signal 34A is shown passing through a skin layer from 1.0 cm to about 1.1 cm, a sub-dermal layer from about 1.1 cm to abut 1.4 cm, a fat layer from about 1.4 cm to about 1.9 cm, and a muscle layer from about 1.9 cm to about 3.9 cm, where the transmitted signal 34A is shown reflecting off of the bone layer resulting in the baseline reflected signal 34B. The reflected signal 34B then passes through the other layers and experiences various delays and attenuations based on the type and positions of material in each layer. It should be noted that FIG. 5 shows only one reflected signal, but this is done for purpose of clarity and skilled technologists will recognize that multiple reflected signals may be received and analyzed.

The baseline reflected signal 34B has a time of flight of about 1.3 nanoseconds to progress through the different layers from transmission to reception. At a later time, a second measurement is made and a transmitted signal 32A is directed into the same region for a subsequent measurement. The transmitted signal 32A is then reflected off the bone layer and is received by the measurement system 100. In this second measurement, the transmitted signal 32A and the reflected signal 32B are delayed substantially compared to the baseline transmitted signal 34A and reflected signal 34B. In this case the round trip time of flight is about 1.5 nanoseconds for the signals 32A and 32B compared to 1.3 nanoseconds for the baseline signals 34A and 34B. In addition to the delay in the signal, the amplitude of the signal may also be different (not shown in FIG. 5) which may also be analyzed to identify possible sources of attenuation.

As discussed above, other energy forms may also be used in the measurement system 100, depending on the embodiment. Other energy forms that may be used by the measurement system 100 to evaluate the tissues will now be discussed. These other forms of measurement may be used to help correlate changes in the UWB measurement to actual and specific structural changes of interest in underlying tissue. In some embodiments of the invention, a single detection instrument such as a patch or handheld device includes electrodes, transducers, antennas, etc. and control circuitry for both UWB interrogation and interrogation with one or more other energy forms that provide complementary tissue content or structure information. In these and similar embodiments using multiple modalities, methods of measuring structural change may comprise deriving a plurality of data sets for both the electromagnetic wave interaction and the other from of energy interaction. Changes in both data sets are detected, from which structural changes can be detected.

High Frequency Electromagnetic Radiation

Electromagnetic radiation at higher frequencies, such as terahertz radiation, or higher, e.g. infrared light, could potentially be used to monitor changes in superficial tissue structure and content. This may be achieved by monitoring the amplitude of reflected radiation from the tissue surface. Any changes in the structure or content (such as edema) can be detected because of absorption by water in the tissues.

Acoustic Radiation

Although acoustic waves are mechanical waves, similar wave propagation principles could be used to detect changes in tissue structure and content. The speed of the acoustic wave in any material is related to temperature, the elastic properties of the material, and the material's density. Thus, any changes in the constituents of the tissue due to its changing physiology or morphology that are reflected by a change of its properties can be detected by using acoustic waves. In one embodiment, the invention comprises equipment configured to focus acoustic waves comprising frequencies in a range from about 1 kHz to about 100 kHz (high bandwidth short pulses or high bandwidth longer time signals composed of multiple frequencies like “chirps”) on tissues and monitor the transmitted and the reflected waves from the tissue. Depending on the type of application, three different methods of analyses are possible, a) Wave attenuation while passing through the tissue; b) Attenuation of reflected waves; and c) Phase difference between transmitted and reflected waves after reflection of a boundary, such as between bone and tissue. Additionally, any temperature changes inside the tissue due to inflammation or an infection response could be tracked because the speed of sound changes with temperature.

The method and devices for applying acoustic signals to one or more regions of the body are well known to those skilled in the art of acoustic signal generation and interpretation, including sonography. It will be appreciated, however, that ultrasound imaging is not necessary to perform the desired function in assessing content or other changes that correlate (either positively or negatively) with the structural changes of interest. In fact, ultrasound images would require complex and expensive equipment that is generally not required for use with the present methods.

Bioelectric Impedance Signals

In another embodiment of the invention, bioelectric impedance of the tissues of interest can be used to monitor the changes in tissue structure and content. This is achieved by passing electrical current through the tissue of interest and measuring the voltage drop across the tissue (or by exciting the tissue with a sinusoidal voltage and measuring the current through the tissue) and calculating the impedance of the tissue to the current flow. Changes in the amplitude of the measured signal as well as the phase difference between the voltage and current signals depend on the tissue properties. As the tissue structure changes (e.g., due to scar tissue growth, tumors etc) and/or content changes (e.g., due to edema, fat/muscle ratio etc), the amplitude of the impedance as well as the phase with respect to the current or voltage excitation change and can be monitored. Additionally, DC signals could also be used for obtaining the changes in the resistance of the tissue. For bio-electrical impedance, focusing of energy (or controlling the volume of measurement) may be achieved by changing the geometry of the electrodes used for driving the current and measuring the voltage. The method and devices for delivering and receiving electrical signals to one or more regions of the body, including necessary electrodes and circuitry, are well known to those skilled in the art of bioelectric impedance. Reference may be made to U.S. Publication Number 2005/0070778, which is hereby incorporated by reference in its entirety.

General Use

The measurement and storage of energy pulses or signals of any frequency comprise a data set which may include reference to time of measurement and/or location of measurement. Such measurement and storage activities may include transformation of the raw data. Such transformations may be useful, allowing facilitated storage of the data set, e.g. data compression, or otherwise facilitate transmission and/or analysis of the data set by the comparator. Signals for use by the comparator may be from one or more of applied energy sources, e.g. radiofrequency, acoustic, electrical or optical. In addition, these signals may be utilized in various combinations over time to provide greater insight into dynamically changing body regions or tissues, e.g. inspection of suspected tumors may be first registered using forms of radiofrequency energy detecting the presence of tissue of differing density. Subsequent observations may include impedance measurements to gauge the increased swelling, blood flow or edema around this site to more accurately provide a trend analysis of change over time.

In some embodiments of the invention, fiducial marker aids, signal alignment aids and/or signal improvement aids may be employed. These aids may include the use of mapping of body regions using electromagnetic signals or other techniques, e.g. MRI, to establish points of reference within data sets or provide aids to more precisely position the measurement device on the body. Employment of these points of reference thereby improves alignment of data sets enabling change in the target region to be more precisely determined. In alternate forms, these aids may include the use of passive or active devices affixed to or implanted within the mammalian body. These aids may provide reference signals or otherwise serve as landmarks to target the measurement device and/or data set. The fiduciary aids may include, but are not limited to: optical alignment aids, e.g. tattoos; signal reflective aids, e.g. implanted metal reflectors or conductive inks; inductively charged implanted radiofrequency transceivers; or implanted acoustic transmitters. Such aids may be arranged in geometric patterns, e.g. cross-hatched, to improve both interpretation of on-body position, e.g. signal alignment, and signal complexity in a known fashion through a three dimensional space to aid subsequent comparator activities.

In certain other related embodiments of the invention, materials either implanted or positioned about the inspected region, may be utilized to aid in the measurement process. For instance, these materials may be utilized to focus the electrical/electromagnetic/acoustic waves through regions of interest or may serve as a highly reflective target behind the region of interest, thereby increasing the effective signal strength of the applied electrical/electromagnetic/acoustic waves.

In other embodiments of the invention, the transmitter elements of the measurement device may have an identity assigned to it. Such identity may include the ability to determine antenna geometries and transmission frequencies. Likewise, other portions of electronic circuitry may have additional identities assigned to the remaining components of the circuitry. Such identities may be useful for enabling construction of disposable and reusable assemblies within the device and allow tracking of said assemblies. Also, such identities allow subsequent identification of the use of the device and form of the device in managing the data sets and coordinating findings of the comparator to the individual measured subject. In addition, such identities may have use in the assignment of encryption keys or other needs for secure transmission of information and assignment/display of the measured data sets.

Use and Applications

In use, the measurement device or devices may be affixed to the subject or positioned about the subject and the device activated by means of a switch or other form of activation. The activation may take place prior to affixing the measurement device to the user. Such activation may also include the use of aids or other alignment tools to ensure correct positioning of measurements. Activation may yet further include activation by means of a switch or other means of a local data collection unit in wireless communication with the measurement device. In such embodiments, an identifier, e.g. a code or serial number, may be used to identify the measurement device to the data collection unit. Such identifiers may include further identifiers detailing the specification of one or more of the measurement sensors. Such identifiers may be transmitted automatically to the data collection unit upon activation or in alternate applications, may be input manually into the data collection unit.

In other embodiments, a single data collection unit may be in communication with a plurality of measurement devices located on or associated with more than one user. In such embodiments, the activation may include an identification means allowing identification of the user in addition to identification of the measurement device.

Upon activation, the measurement device may periodically or upon command obtain measured data from one or more body regions. The monitoring period per data set may be seconds, e.g. every second, or longer, dependent upon the nature of the bioparameter being measured for change. In other embodiments, the monitoring frequency may automatically adjust, dependent upon the rate of change in the desired portion of the measured region. In addition, different devices may have different configurations of sensors and/or different monitoring frequencies applied to the same subject. The measured data may be supplied directly to the comparator for analysis or it may be processed in some form prior to being supplied to the comparator for determination of change and/or magnitude of such change.

Applications of electromagnetic or other forms of energy waves for monitoring of change of one or more regions of a mammalian body include, but are not limited to:
Monitoring for change in body hydration and/or electrolyte levels over time, including detection of detrimental levels of systemic hydration, projection of future hydration status and recovery or return to acceptable levels of fluid and/or total ion composition levels of bodily fluids.
Monitoring for jugular vein distension associated with hypertension such as period measurements separated by intervals shorter than the determined heart rate to permit assessment of the magnitude of vascular distension (change) due to blood pressure.
Monitoring of internal organs, including heart, kidney or liver for changes such as fluid infiltration/local edema associated with organ failure and/or allograft rejection.
Monitoring of ovarian development/folliculogenesis during the menstrual cycle to aid in the determination of proper timing for the administration of drugs associated with fertility and/or pregnancy.
Monitoring of wounds and/or scar tissue associated with wounds to aid in the detection of infection, impaired healing or to guide timing of wound treatments, e.g. debridement or for wound staging to determine the extent of deep tissue injury.
Monitoring of body locations, e.g. sacrum, hips or heels, to detect changes in underlying tissue fluid status associated with the pre-emergence of ulcers or other forms of skin/tissue disease states.
Monitoring of fluid build-up or change in internal body compartments, organs or muscle groups, e.g. internal hemorrhage or compartment syndrome, associated with disease state, trauma or surgical interventions to allow more effective detection and subsequent therapeutic response.
Detection of fluid build-up over time associated with the onset and/or progression of cardiogenic or non-cardiogenic pulmonary edema.
Detection of body composition change, e.g. change in muscle composition associated with wasting diseases such as HIV disease progression or muscular dystrophy caused by neuromuscular disorders.
Monitoring stenosis or occlusion of blood vessels, or implanted vascular devices e.g. venous grafts.

Additional applications may also include providing a user of the device with quantitative feedback regarding the magnitude of the measured change and any periodic nature to this change, e.g. time of day, such that the user may self-medicate in order to relieve the symptoms or otherwise take some form of therapeutic action associated with the change in the underlying bioparameter. Alternatively, the use of remote data management systems receiving data from one or more data collection units may permit clinician adjusted therapy changes from a remote location upon review of the data sets and output of comparator activities.

While the above detailed description has shown, described, and pointed out novel features of the invention as applied to various aspects, it will be understood that various omissions, substitutions, and changes in the form and details of the device or process illustrated may be made by those skilled in the art without departing from the scope of this disclosure. As will be recognized, the invention may be embodied within a form that does not provide all of the features and benefits set forth herein, as some features may be used or practiced separately from others. The scope of this disclosure is defined by the appended claims, the foregoing description, or both. All changes which come within the meaning and range of equivalency of the claims are to be embraced within their scope.

What is claimed is:

1. A method of detecting a physiological condition in a portion of a subject; the method comprising:
applying electromagnetic waves having frequencies within a range of about 1 GHz to about 100 GHz to a region of the subject at a plurality of points in time;
deriving a plurality of data sets from measured parameters of interaction between the region of the subject and the electromagnetic waves at the plurality of points in time, wherein the content of the data sets is dependent at least in part on tissue structure in the region at the plurality of points in time;
comparing the plurality of data sets to detect a change over time in the interaction between the region of the subject and the electromagnetic waves; and
detecting a change in tissue structure in the region of the subject based at least in part on the comparing.

2. The method of claim 1, wherein the electromagnetic waves comprise ultra wideband radio signals.

3. The method of claim 1 wherein the electromagnetic waves comprise signals of frequency bands other than ultra wideband radio signals.

4. The method of claim 1, wherein detecting the change in tissue structure comprises detecting one or more of scar tissue growth, tumors, and fractures.

5. The method of claim 1, wherein said detected change is associated with a healing wound.

6. The method of claim 1, where said detected change is associated with compartment syndrome.

7. The method of claim 1, wherein said detected change is a change in transplant organ status.

8. The method of claim 1, wherein said detected change is change in ovary dimension.

9. The method of claim 1, further comprising employing fiducial points or alignment aids.

10. A method of determining detecting a physiological condition in a portion of a subject; the method comprising:
applying electromagnetic waves residing within a frequency range from about 1 GHz to about 100 GHz to a region of the subject at a plurality of points in time;
applying a different form of energy to the region of the subject at a plurality of points in time;
deriving a first plurality of data sets from measured parameters of interaction between the region of the subject and the electromagnetic waves at the plurality of points in time;
deriving a second plurality of data sets from measured parameters of interaction between the region of the subject and the different form of energy at the plurality of points in time;
comparing the first plurality of data sets to detect a change over time in the interaction between the region of the subject and the electromagnetic waves;
comparing the second plurality of data sets to detect a change over time in the interaction between the region of the subject and the different form of energy; and
detecting a change in tissue structure in the region of the subject based at least in part on the comparing.

11. The method of claim 10, wherein the other form of energy is acoustic energy.

12. The method of claim 10, wherein the other form of energy is electrical energy.

13. The method of claim 10, wherein the other form of energy is sensitive to chemical content of the region.

14. The method of claim 13, wherein the other form of energy is sensitive to hydration of the region.

15. A system for measuring change over time in tissue structure in a body region, the system comprising:
at least one antenna and circuitry coupled thereto configured to transmit and receive electromagnetic wave signals;
circuitry configured to convert said signals into one or more data sets; and
a comparator subsystem for performing mathematical calculations on at least two data sets for the determination of change in the tissue structure of the measured body region.

16. The system of claim 13, wherein said comparator is located in a separate data collection unit in wireless communication with said antenna and circuitry.

17. The system in claim 13, wherein the antenna and circuitry for measurement of tissue changes is also configured for data communication to remote data collection units.

18. A system for measuring change over time in tissue structure in a body region, the system comprising:
an applicator of about 1 GHz to about 100 GHz electromagnetic energy;
a detector configured to detect parameters of interaction between the tissue and the electromagnetic energy;
an applicator of another form of energy;
a detector configured to detect parameters of interaction between the tissue and the other form of energy;
wherein the applicators are housed or supported by a common structure.

19. The system of claim 18, wherein the common structure comprises a patch.

20. The system of claim 18, wherein the common structure comprises a handheld unit.
